(19) United States
(12) Patent Application Publication
(10) Pub. No.: US 2025/0009682 A1
(43) Pub. Date: Jan. 9, 2025

(54) PATCH

(71) Applicant: KANEKA CORPORATION, Osaka (JP)

(72) Inventors: Hiroyuki OGINO, Hyogo (JP); Masaoki GOTO, Hyogo (JP)

(21) Appl. No.: 18/710,056

(22) PCT Filed: Nov. 24, 2022
(86) PCT No.: PCT/JP2022/043366

§ 371 Date: May 14, 2024

(30) Foreign Application Priority Data
Dec. 2, 2021 (JP) 2021-196430
Nov. 9, 2022 (JP) 2022-179851

(51) Int. Cl. A61K 9/70; A61K 31/167; A61K 31/245; A61K 47/02; A61K 47/20; A61K 47/22; A61K 47/44; A61K 47/54

(52) U.S. Cl. CPC A61K 9/7053; A61K 31/167; A61K 31/245; A61K 47/02; A61K 47/20; A61K 47/22; A61K 47/44; A61K 47/543

(57) ABSTRACT

A patch includes a support and an adhesive layer on the support. The adhesive layer includes a local anesthetic, a thermoplastic elastomer, a higher fatty acid ester, and dimethyl sulfoxide.

### TECHNICAL FIELD

The present invention relates to a patch including a local anesthetic.

### BACKGROUND

Local anesthetic preparations for patches are widely used for reducing pain during medical procedures, such as punctures by syringe needles or peripheral intravenous catheters, minor skin operations, and the like. In Japan, a lidocaine tape (product name: Painlesstape) is commercially available, and an accompanying instruction manual discloses a usage method such that a tape should be applied to a target puncture site for approximately 30 minutes, when the product is used to reduce pain during a puncture by a peripheral intravenous catheter. However, a level of anesthesia obtained by the described method is insufficient, and there is a report that an application time of approximately 100 minutes is needed to achieve a sufficient pain relief effect (Non-Patent Literature 1).

A local anesthetic cream (EMLA CREAM) including a mixture of lidocaine and prilocaine as an active ingredient is also commercially available in Japan. The stronger anesthetic potency of the cream compared with the anesthetic potency of the lidocaine tape is acknowledged (Patent Literature 1).

Patent Literature 2 discloses a technology of a patch including a mixture of lidocaine and prilocaine.

Patent Literature 3 discloses a technology of a patch using a higher fatty acid ester as a plasticizer for achieving a rapid onset of anesthesia.

### CITATION LIST

### Patent Literature

Patent Literature 1: Japanese Patent Application Laid-Open (JP-A) No. 54-101414
Patent Literature 2: Japanese Patent No. (JP-B) No. 6808628
Patent Literature 3: International Publication No. WO 2020/184208

### Non-Patent Literature

Non-Patent Literature 1: YAKUGAKU ZASSHI 127(11) 1797-1799

### SUMMARY OF THE INVENTION

### Technical Problem

EMLA CREAM disclosed in Patent Literature 1 has problems such that procedures are complicated and involve many processes. Specifically, the cream is spread on skin as a thick lump, and the applied cream needs to be sealed with a film or the like by an occlusive dressing technique (ODT), or the used cream needs to be completely wiped off by a patient or medical practitioner after use. Moreover, a potential risk for skin damages caused during spreading the cream on skin or wiping the cream off from skin is concerned. Furthermore, there are problems associated with remaining stickiness or deposition of the cream on clothes or the like. Therefore, there has been a growing demand for application of a local anesthetic on patches, which can be used in a simple method. However, patches that achieve rapid onset of anesthesia have not been known at all. Patches, which may give a relatively low level of anesthesia in a short period, but achieve rapid onset of anesthesia, have been strongly desired. The technology disclosed in Patent Literature 2 provides the data demonstrating excellent skin permeability of the drug. However, EMLA CREAM is not used as a control, and there is no disclosure of a comparison with the same active ingredient. In addition, local anesthetic potency, which is a crucial point, is not mentioned therein. In Patent Literature 3, the local anesthetic is limited to tetracaine, and data of a mixture of lidocaine and prilocaine is not disclosed. Accordingly, the present invention aims to solve the above-described various problems existing in the related art, and to provide a patch that can be used in a simple method and achieves rapid onset of anesthesia as a medical product, and a production method thereof.

### Solution to Problem

The present inventors have accomplished the present invention based on the following insight. When a patch includes an adhesive layer including a local anesthetic, and the adhesive layer includes at least a thermoplastic elastomer, a higher fatty acid ester, and dimethyl sulfoxide, the patch, which serves as a local anesthetic patch that can be used in a simple method, achieves rapid onset of anesthesia.

The present invention has been accomplished based on the insights of the present inventors, and means for solving the above-described problems are as follows.

<1> A patch includes a support and an adhesive layer on the support, where the adhesive layer includes a local anesthetic, a thermoplastic elastomer, a higher fatty acid ester, and dimethyl sulfoxide.

<2> A method for producing the patch according to <1> includes mixing the local anesthetic, the thermoplastic elastomer, the higher fatty acid ester, and the dimethyl sulfoxide.

### Advantageous Effects of Invention

According to the present invention, a patch that can be used in a simple method and achieves rapid onset of a sufficient level of anesthesia, and a production method thereof can be provided.

### DETAILED DESCRIPTION OF THE INVENTION

### (Patch)

The patch includes a support and an adhesive layer on the support. The patch may further include other elements.

An amount of a volatile solvent, which is used in the below-described mixing step, remaining in the adhesive layer is preferably 0.5% by mass or less, and more preferably 0.1% by mass or less, relative to 100% by mass of a total amount of constituent components of the adhesive layer.

### <Support>

The support is not particularly limited, and may be appropriately selected according to the intended purpose. Examples of the support include supports generally used for adhesive sheets to adhere to skin or for transdermal preparations.

A material of the support is not particularly limited, and may be appropriately selected according to the intended purpose. Examples of the material of the support include: polyesters, such as polyethylene terephthalate and the like; polyolefins, such as polyethylene, polypropylene, and the like; polyurethane; ethylene-vinyl acetate copolymers; polyvinyl chloride; and the like.

A structure of the support may be a single-layer structure or a multiple-layer structure. Moreover, the support may be of knitted fabrics, woven fabrics, nonwoven fabrics, a film, a foam, a porous body, a network structure, a sheet, or a flat plate.

In order to minimize accumulation of static electricity within the support, an antistatic agent may be added to the woven fabrics, nonwoven fabrics, film, or the like that constitutes the support. Moreover, nonwoven fabrics, woven fabrics, or knitted fabrics, or a laminate of a film with nonwoven fabrics, woven fabrics, or knitted fabrics may be used as the support to achieve suitable anchoring to an adhesive layer.

A thickness of the support is not particularly limited, and may be appropriately selected according to the intended purpose. A thickness of the film is preferably 10 μm or greater and 100 μm or less, and more preferably 15 μm or greater and 50 μm or less. A thickness of a porous sheet, such as the knitted fabrics, the woven fabrics, the nonwoven fabrics, the foam support, and the like, is preferably 50 μm or greater and 2,000 μm or less, and more preferably 100 μm or greater and 1,000 μm or less.

### <Adhesive Layer>

The adhesive layer includes (a) a local anesthetic, (b) a thermoplastic elastomer, (c) a higher fatty acid ester, and (d) dimethyl sulfoxide, and may further include other components.

### (a) Local Anesthetic

The “local anesthetic” is not particularly limited, and may be appropriately selected according to the intended purpose. Examples of the local anesthetic include substances each having a structure having a basic skeleton that includes an aromatic ring, an alkyl chain, and an amino group, where the aromatic ring and the alkyl chain are bonded via an ester bond or an amide bond. Examples of the substances having the above-described structure include lidocaine, prilocaine, tetracaine, benzocaine, bupivacaine, mepivacaine, levobupivacaine, ropivacaine, and the like. Among the above-listed examples, lidocaine, prilocaine, tetracaine, or benzocaine is preferred, and lidocaine or prilocaine is more preferred. The above-listed local anesthetics may be used alone or in combination.

Moreover, the local anesthetic may be a free base or a pharmaceutically acceptable salt, and is not particularly limited. The pharmaceutically acceptable salt is not particularly limited, and may be an inorganic salt or an organic salt. Examples of the inorganic salt include hydrochlorides, hydrobromides, nitrates, sulfates, phosphates, and the like. Examples of the organic salt include formates, acetates, trifluoroacetates, propionates, lactates, tartrates, oxalates, fumarates, maleates, citrates, malonates, methanesulfonates, and the like.

The above-listed the pharmaceutically acceptable salts may be used alone or in combination. Moreover, a mixture of a free base and a salt may be used.

Among the above-listed examples, a free base or hydrochloride is preferred in view of easiness in sourcing, and a free base is more preferred in view of dispersibility in an adhesive.

When the two or more local anesthetics are used in combination, lidocaine and a local anesthetic other than lidocaine (may be also referred to as lidocaine and another local anesthetic) are preferably used in combination. The prilocaine, tetracaine, benzocaine, bupivacaine, mepivacaine, levobupivacaine, ropivacaine, or the like is a solid at room temperature, but a melting point thereof is reduced to transform the solid state to a liquid state at room temperature when mixed with the solid lidocaine.

A mixture including the lidocaine and another local anesthetic is not particularly limited, and may be appropriately selected according to the intended purpose. A mixture including lidocaine and prilocaine, a mixture including lidocaine and tetracaine, or a mixture including lidocaine and benzocaine is preferred, and a mixture including lidocaine and prilocaine is more preferred.

The lower limit of an amount of the local anesthetic in the adhesive layer (a proportion of the local anesthetic to 100% by mass of a total amount of constituent components of the adhesive layer) is not particularly limited, and may be appropriately selected according to the intended purpose. In view of assuring solubility of the local anesthetic in the adhesive layer and desired skin permeability, the lower limit is preferably 1% by mass or greater, more preferably 2% by mass or greater, yet more preferably 3% by mass or greater, yet even more preferably 5% by mass or greater, particularly preferably 6% by mass or greater, and the most preferably 7% by mass or greater.

The upper limit of an amount of the local anesthetic in the adhesive layer (a proportion of the local anesthetic to 100% by mass of a total amount of constituent components of the adhesive layer) is not particularly limited, and may be appropriately selected according to the intended purpose. The upper limit is preferably 40% by mass or less, more preferably 30% by mass or less, yet more preferably 20% by mass or less, and yet even more preferably 15% by mass or less.

When the two or more local anesthetics are used in combination, the lower limit of an amount of the mixture of the local anesthetics in the adhesive layer (a proportion of a sum of the amounts of the local anesthetics to 100% by mass of a total amount of constituent components of the adhesive layer) is not particularly limited, and may be appropriately selected according to the intended purpose. In view of assuring solubility of the mixture in the adhesive layer and desired skin permeability, the lower limit is preferably 1% by mass or greater, more preferably 2% by mass or greater, yet more preferably 3% by mass or greater, yet even more preferably 5% by mass or greater, particularly preferably 10% by mass or greater, and the most preferably 13% by mass or greater.

When the two or more local anesthetics are used in combination, the upper limit of an amount of the mixture of the local anesthetics in the adhesive layer (a proportion of a sum of the amounts of the local anesthetics to 100% by mass of a total amount of constituent components of the adhesive layer) is not particularly limited, and may be appropriately selected according to the intended purpose. The upper limit is preferably 40% by mass or less, more preferably 30% by mass or less, yet more preferably 20% by mass or less, and yet even more preferably 15% by mass or less.

When the lidocaine and another local anesthetic are used as the local anesthetic, the lower limit of an amount of the lidocaine in the mixture including the lidocaine and another local anesthetic is not particularly limited, and may be appropriately selected according to the intended purpose. The lower limit of the amount of the lidocaine is preferably 30% by mass or greater, more preferably 40% by mass or greater, yet more preferably 45% by mass or greater, and yet even more preferably 50% by mass or greater.

When the lidocaine and another local anesthetic are used as the local anesthetic, the upper limit of the amount of the lidocaine in the mixture including the lidocaine and another local anesthetic is not particularly limited, and may be appropriately selected according to the intended purpose. The upper limit of the amount of the lidocaine is preferably 70% by mass or less, more preferably 60% by mass or less, and yet more preferably 55% by mass or less.

The most preferably, a mass ratio of the lidocaine to another local anesthetic in the mixture of the lidocaine and another local anesthetic is 50/50 (mass ratio).

### (b) Thermoplastic Elastomer

The “thermoplastic elastomer” is an elastomer exhibiting thermoplasticity, where the elastomer is softened to exhibit fluidity when heat is applied, and is returned back to a rubber-like elastic body when cooled. Examples of the thermoplastic elastomer include various thermoplastic elastomers, such as urethane-based thermoplastic elastomers, acrylic-based thermoplastic elastomers, styrene-based thermoplastic elastomers, olefin-based thermoplastic elastomers, silicone-based thermoplastic elastomers, and the like.

The urethane-based thermoplastic elastomers mean that elastomers are formed of various polymers each having a polyurethane skeleton. The acrylic-based thermoplastic elastomers mean that elastomers are formed of various acrylic polymers each including a polyacrylic acid ester and/or a polymethacrylic acid ester as a skeleton. The styrene-based thermoplastic elastomers mean that elastomers are formed of various polymers each having a polystyrene skeleton. The olefin-based thermoplastic elastomers mean that elastomers are formed of various polymers each having a polyolefin skeleton. The silicone-based thermoplastic elastomers mean that elastomers are formed of various polymers each having a silicone skeleton.

Among the above-listed examples, styrene-based thermoplastic elastomers are preferred, and a styrene-based block copolymer is more preferred in view of achieving both adequate adhesion to skin and low skin irritability, which are objects of the present invention.

The styrene-based block copolymer is not particularly limited, and may be appropriately selected according to the intended purpose. Examples of the styrene-based block copolymer include styrene-butadiene block copolymers, styrene-butadiene-styrene block copolymers, styrene-isoprene block copolymers, styrene-isoprene-styrene block copolymers, styrene-ethylene/butylene block copolymers, styrene-ethylene/butylene-styrene block copolymers, styrene-ethylene/propylene block copolymers, styrene-ethylene/propylene-styrene block copolymers, styrene-isobutylene block copolymers, styrene-isobutylene-styrene block copolymers, and the like.

The “ethylene/butylene” encompasses a copolymer block of ethylene and butylene. The “ethylene/propylene” encompasses a copolymer block of ethylene and propylene. The above-listed styrene-based block copolymers may be used alone or in combination.

Among the above-listed styrene-based block copolymers, at least one or two or more selected from the group consisting of styrene-isoprene-styrene block copolymers and styrene-isoprene block copolymers are particularly preferred, and a mixture including a styrene-isoprene-styrene block copolymer and a styrene-isoprene block copolymer is more preferred in view of availability or handling of products for patches, as well as achieving both adequate adhesion to skin and a reduction of adhesive residues through improvement of cohesion of an adhesive layer.

When a mixture including the styrene-isoprene-styrene block copolymer and the styrene-isoprene block copolymer is used as the styrene-based block copolymer, the lower limit of an amount of the styrene-isoprene block copolymer in the mixture is not particularly limited, and may be appropriately selected according to the intended purpose. In view of assuring adequate adhesion, the lower limit of the amount of the styrene-isoprene block copolymer in the mixture is preferably 15% by mass or greater, more preferably 20% by mass or greater, yet more preferably 30% by mass or greater, particularly preferably 40% by mass or greater, and the most preferably 50% by mass or greater.

When the mixture including the styrene-isoprene-styrene block copolymer and the styrene-isoprene block copolymer is used as the styrene-based block copolymer, the upper limit of the amount of the styrene-isoprene block copolymer in the mixture is not particularly limited, and may be appropriately selected according to the intended purpose. In view of assuring adequate cohesion, the upper limit of the amount of the styrene-isoprene block copolymer in the mixture is preferably 80% by mass or less.

An amount of the styrene in the styrene-isoprene-styrene block copolymer is not particularly limited, and may be appropriately selected according to the intended purpose. The lower limit of the amount of the styrene is preferably 5% by mass or greater, and more preferably 10% by mass or greater. The upper limit of the amount of the styrene is preferably 60% by mass or less, and more preferably 50% by mass or less. Among the above-listed examples, the amount of the styrene is preferably 5% by mass or greater and 60% by mass or less, and more preferably 10% by mass or greater and 50% by mass or less. Moreover, a weight average molecular weight of the styrene-isoprene-styrene block copolymer as measured by gel permeation chromatography (GPC) is not particularly limited, and may be appropriately selected according to the intended purpose. The weight average molecular weight of the styrene-isoprene-styrene block copolymer is preferably 20,000 or greater and 500,000 or less, and more preferably 30,000 or greater and 300,000 or less.

Moreover, an amount of the styrene in the styrene-isoprene block copolymer is not particularly limited, and may be appropriately selected according to the intended purpose. The lower limit of the amount of the styrene is preferably 5% by mass or greater, and more preferably 10% by mass or greater. The upper limit of the amount of the styrene is preferably 50% by mass or less, and more preferably 40% by mass or less. Among the above-listed examples, the amount of the styrene is preferably 5% by mass or greater and 50% by mass or less, and more preferably 10% by mass or greater and 40% by mass or less. Moreover, a weight average molecular weight of the styrene-isoprene block copolymer as measured by GPC is not particularly limited, and may be appropriately selected according to the intended purpose. The weight average molecular weight of the styrene-isoprene block copolymer is preferably 10,000 or greater and 500,000 or less, and more preferably 20,000 or greater and 300,000 or less.

A viscosity of the styrene-based block copolymer is not particularly limited, and may be appropriately selected according to the intended purpose. In view of a desirable balance of adhesion physical properties, the lower limit of a viscosity of a 25% by mass toluene solution of the styrene-based block copolymer at a solution temperature of 25° C. is preferably 500 mPa·s or greater, more preferably 800 mPa·s or greater, and yet more preferably 900 mPa·s or greater. The upper limit of the viscosity is preferably 2000 mPa·s or less, more preferably 1800 mPa·s or less, and yet more preferably 1500 mPa·s or less. Note that, the “viscosity of the 25% by mass toluene solution at 25° C.” is a value measured according to a viscosity measuring method for a styrene-isoprene-styrene block copolymer described in page 395 of “Japanese Pharmaceutical Excipients 2013” (published by Yakuji Nippo, Limited).

As each of the styrene-isoprene-styrene block copolymer and the styrene-isoprene block copolymer, a copolymer produced by any method available in the related art may be used. As each of the styrene-isoprene-styrene block copolymer and the styrene-isoprene block copolymer, a commercially available product satisfying the above-described properties may be used. Moreover, the mixture including the styrene-isoprene-styrene block copolymer and the styrene-isoprene block copolymer is also commercially available. A commercially available product of the mixture in which the styrene-isoprene-styrene block copolymer satisfying the above-described properties and the styrene-isoprene block copolymer satisfying the above-described properties are mixed at the above-described blending ratio may be suitably used.

Examples of the commercial products include: “KRATON D1111,” “KRATON D1163,” “KRATON D1113,” and “KRATON D1119,” which are produced by KRATON POLYMERS; “JSR SIS5229,” “JSR SIS5002,” “JSR SIS5403,” and “JSR SIS5505,” which are produced by JSR Corporation; “Quintac 3421,” “Quintac 3433N,” “Quintac 3520,” “Quintac 3450,” and “Quintac 3270,” which are produced by Zeon Corporation; and the like. Among the above-listed examples, “KRATON D1163,” “KRATON D1113,” “JSR SIS5403” “JSR SIS5505,” “Quintac 3433N,” and “Quintac 3520” are preferred, and “JSR SIS5505” and “Quintac 3520” are more preferred, in view of a blending ratio between the triblock copolymer and the diblock copolymer, and a solution viscosity.

An amount of the thermoplastic elastomer in the adhesive layer (a proportion of the thermoplastic elastomer to 100% by mass of a total amount of constituent components of the adhesive layer) is not particularly limited, and may be appropriately selected according to the intended purpose. If the amount of the thermoplastic elastomer in the adhesive layer is too small, it may be difficult to maintain a shape of the adhesive layer. If the amount of the thermoplastic elastomer in the adhesive layer is too large, adhesion to skin may become inadequate. Therefore, the lower limit of the amount of the thermoplastic elastomer is preferably 20% by mass or greater, more preferably 25% by mass or greater, and yet more preferably 30% by mass or greater. The upper limit of the amount of the thermoplastic elastomer is preferably 70% by mass or less, more preferably 65% by mass, and yet more preferably 60% by mass or less. Among the above-listed examples, the amount of the thermoplastic elastomer is preferably 20% by mass or greater and 70% by mass or less, more preferably 25% by mass or greater and 65% by mass or less, and yet more preferably 30% by mass or greater and 60% by mass or less.

### (c) Higher Fatty Acid Ester

The “higher fatty acid ester” is a compound in which a carboxyl group of a higher fatty acid forms an ester bond with an aliphatic alcohol. The higher fatty acid ester has an effect of plasticizing the thermoplastic elastomer to an appropriate level, thereby contributing to adhesion. Moreover, the higher fatty acid ester has an appropriate level of affinity with the local anesthetic, thus the higher fatty acid ester also contributes to improvement of solubility of the local anesthetic or inhibition of crystallization.

A higher fatty acid constituting the higher fatty acid ester may be a straight-chain higher fatty acid or a branched-chain higher fatty acid. Moreover, the higher fatty acid may be a saturated higher fatty acid or an unsaturated higher fatty acid. In view of a plasticizing effect and thermal stability of the thermoplastic elastomer, the higher fatty acid is preferably a saturated higher fatty acid.

The lower limit of a carbon number of the higher fatty acid (a carbon number of an ester moiety of the higher fatty acid ester) is not particularly limited, and may be appropriately selected according to the intended purpose. In view of cohesion, the lower limit of the carbon number is preferably 12 or greater, more preferably 14 or greater, and yet more preferably 16 or greater.

The upper limit of the carbon number of the higher fatty acid (the carbon number of an ester moiety of the higher fatty acid ester) is not particularly limited, and may be appropriately selected according to the intended purpose. In view of solubility of the drug, the upper limit of the carbon number is preferably 30 or less, more preferably 24 or less, yet more preferably 20 or less, and particularly preferably 16 or less.

Examples of the saturated higher fatty acid include capric acid (carbon number: 10), lauric acid (carbon number: 12), myristic acid (carbon number: 14), palmitic acid (carbon number: 16), stearic acid (carbon number: 18), isostearic acid (carbon number: 18), arachidic acid (carbon number: 20), behenic acid (carbon number: 22), lignoceric acid (carbon number: 24), cerotic acid (carbon number: 26), montanic acid (carbon number: 28), melissic acid (carbon number: 30), and the like. Among the above-listed examples, myristic acid, palmitic acid, or stearic acid is preferred, myristic acid or palmitic acid is more preferred, and myristic acid is yet more preferred, in view of an effect of plasticizing the thermoplastic elastomer.

Examples of the unsaturated higher fatty acid include palmitoleic acid (carbon number: 16), oleic acid (carbon number: 18), linoleic acid (carbon number: 18), (9,12,15)-linolenic acid (carbon number: 18), (6,9,12)-linolenic acid (carbon number: 18), eleostearic acid (carbon number: 18), and the like. Among the above-listed examples, oleic acid or linoleic acid is preferred in view of an effect of plasticizing the thermoplastic elastomer.

The aliphatic alcohol constituting the higher fatty acid ester is preferably a saturated or unsaturated aliphatic alcohol having a carbon number of 1 or greater and 20 or less. Examples thereof include methanol, ethanol, propanol, isopropanol, butanol, hexanol, heptanol, octanol, decanol, cetanol, myristyl alcohol, hexyldecanol, oleyl alcohol, octyldodecanol, and the like.

Specific suitable examples of the higher fatty acid ester include: myristic acid esters, such as isopropyl myristate, ethyl myristate, octyldodecyl myristate, and the like; palmitic acid esters, such as cetyl palmitate, isopropyl palmitate, ethyl palmitate, and the like; stearic acid esters, such as isopropyl stearate and the like; isostearic acid esters, such as hexyldecyl isostearate and the like; 2-ethylhexanoic acid esters, such as cetyl 2-ethylhexanoate and the like; oleic acid esters, such as decyl oleate, octyldodecyl oleate, oleyl oleate, and the like; and linoleic acid esters, such as ethyl linoleate and the like. Among the above-listed examples, myristic acid esters, palmitic acid esters, isostearic acid esters, and 2-ethylhexanoic acid esters are preferred, and octyldodecyl myristate, cetyl palmitate, hexyldecyl isostearate, and cetyl 2-ethylhexanoate are more preferred in view of an effect of plasticizing the thermoplastic elastomer. The above-listed higher fatty acid esters may be used alone, or in combination.

An amount (proportion) of the higher fatty acid ester in the adhesive layer relative to 100 parts by mass of the thermoplastic elastomer is not particularly limited, and may be appropriately selected according to the intended purpose. If the amount of the higher fatty acid ester is too small, desirable adhesion and solubility of the drug may not be obtained. If the amount of the higher fatty acid ester is too large, it may be difficult to maintain a shape of the adhesive layer. Therefore, the lower limit of the amount of the higher fatty acid ester is preferably 25 parts by mass or greater, more preferably 30 parts by mass or greater, yet more preferably 50 parts by mass or greater, particularly preferably 70 parts by mass or greater, and the most preferably 80 parts by mass or greater. The upper limit of the amount of the higher fatty acid ester is preferably 200 parts by mass or less, more preferably 150 parts by mass or less, yet more preferably 100 parts by mass or less, and particularly preferably 90 parts by mass or less. Among the above-listed examples, the amount of the higher fatty acid ester is preferably 25 parts by mass or greater and 200 parts by mass or less, and more preferably 30 parts by mass or greater and 150 parts by mass or less.

An amount of the higher fatty acid ester in the adhesive layer (a proportion of the higher fatty acid ester to 100% by mass of a total amount of constituent components of the adhesive layer) is not particularly limited, and may be appropriately selected according to the intended purpose. In view of solubility of the drug, the lower limit of the amount of the higher fatty acid ester in the adhesive layer is preferably 10% by mass or greater, more preferably 15% by mass or greater, and yet more preferably 20% by mass or greater. If the amount of the higher fatty acid ester is too large, it may be difficult to maintain a shape of the adhesive layer. Therefore, the upper limit of the amount of the higher fatty acid ester in the adhesive layer is preferably 70% by mass or less, more preferably 65% by mass or less, yet more preferably 60% by mass or less, particularly preferably 50% by mass or less, and the most preferably 40% by mass or less. Among the above-listed examples, the amount of the higher fatty acid ester is preferably 10% by mass or greater and 70% by mass or less, more preferably 15% by mass or greater and 65% by mass or less, and yet more preferably 20% by mass or greater and 60% by mass or less.

### (d) Dimethyl Sulfoxide

In the present invention, the dimethyl sulfoxide has an effect of dissolving a local anesthetic and an effect of facilitating skin permeation of a local anesthetic.

An amount (a ratio) of the dimethyl sulfoxide in the adhesive layer relative to 100 parts by mass of the local anesthetic in the adhesive layer is not particularly limited, and may be appropriately selected according to the intended purpose. In view of dissolution of the local anesthetic for inhibiting crystallization and improvement of skin permeability, the lower limit of the amount (ratio) of the dimethyl sulfoxide is preferably 5 parts by mass or greater (1/0.05 or greater), more preferably 10 parts by mass or greater (1/0.1 or greater), yet more preferably 15 parts by mass or greater (1/0.15 or greater), and particularly preferably 20 parts by mass or greater (1/0.2 or greater). The upper limit of the amount (ratio) of the dimethyl sulfoxide is preferably 150 parts by mass or less (1/1.5 or less), more preferably 100 parts by mass or less (1/1 or less), yet more preferably 80 parts by mass or less (1/0.8 or less), yet even more preferably 50 parts by mass or less (1/0.5 or less), particularly preferably 40 parts by mass or less (1/0.4 or less), and the most preferably 30 parts by mass or less (1/0.3 or less).

An amount of the dimethyl sulfoxide in the adhesive layer (a proportion of the dimethyl sulfoxide to 100% by mass of a total amount of constituent components of the adhesive layer) is not particularly limited, and may be appropriately selected according to the intended purpose. If the amount of the dimethyl sulfoxide is too large, cohesion of an adhesive may decrease so that a resulting adhesive layer may not be able to function as an adhesive. Therefore, the lower limit of the amount of the dimethyl sulfoxide is preferably 0.1% by mass or greater, more preferably 0.3% by mass or greater, yet more preferably 0.5% by mass or greater, yet even more preferably 1% by mass or greater, particularly preferably 2% by mass or greater, and the most preferably 3% by mass or greater. The upper limit of the amount of the dimethyl sulfoxide is preferably 20% by mass or less, more preferably 15% by mass or less, yet more preferably 10% by mass or less, and particularly preferably 5% by mass or less.

### Other Components

The above-mentioned other components are not particularly limited, and may be appropriately selected according to the intended purpose. Examples of the above-mentioned other components include (e) a medium-chain triglyceride, (f) a filler, (g1) a tackifier, (g2) a polyhydric alcohol fatty acid monoester, (g3) an alcohol-based solvent, (g4) an ester-based solvent, (g5) an amide-based solvent, (g6) a liquid organic acid, (g7) a carboxylic acid salt, (g8) lactone, (g9) a surfactant, (g10) an antioxidant, (g11) a crystallization inhibitor, (g12) a nonvolatile hydrocarbon oil, and the like.

In view of improving physical properties of the adhesive layer, the patch of the present invention may include the (e) medium-chain triglyceride or the (f) filler.

In view of improvement in adhesion of the adhesive layer, improvement in dispersibility or stability of the local anesthetic in the adhesive layer, and improvement in skin permeability of the local anesthetic, the patch of the present invention may include the (g1) tackifier, the (g2) polyhydric alcohol fatty acid monoester, the (g3) alcohol-based solvent, the (g4) ester-based solvent, the (g5) amide-based solvent, the (g6) liquid organic acid, the (g7) carboxylic acid salt, the (g8) lactone, the (g9) surfactant, the (g10) antioxidant, the (g11) crystallization inhibitor, or the (g12) nonvolatile hydrocarbon oil.

### (e) Medium-Chain Triglyceride

The medium-chain triglyceride is a triglyceride formed between a fatty acid having a carbon number of 6 or greater and 12 or less, such as caproic acid, caprylic acid, capric acid, lauric acid, or the like, and glycerin. In the present invention, caprylic acid triglyceride that is a liquid at room temperature, a mixture of caprylic acid triglyceride and capric acid triglyceride, a mixture of caprylic acid triglyceride, capric acid triglyceride, and lauric acid triglyceride, or the like may be used as the medium-chain triglyceride. Moreover, oil or fat that includes a large amount of any of the foregoing and is a liquid at room temperature may be also used. Examples of the oil or fat include olive oil, almond oil, safflower oil, soybean oil, corn oil, sesame oil, coconut oil, orange oil, ginger oil, orange peel oil, rapeseed oil, castor oil, sunflower oil, cottonseed oil, peanut oil, and the like. Among the above-listed examples, sesame oil is preferred. The above-listed medium-chain triglycerides may be used alone or in combination.

The dimethyl sulfoxide is a liquid having a melting point of 19° C. When the dimethyl sulfoxide is used as an additive, the dimethyl sulfoxide may be frozen (crystallized) in the adhesive layer during storage in a cold place. The medium-chain triglyceride has an effect of inhibiting crystallization of the dimethyl sulfoxide.

When the adhesive layer includes the medium-chain triglyceride, the lower limit of an amount of the medium-chain triglyceride in the adhesive layer is not particularly limited, and may be appropriately selected according to the intended purpose. In view of an effect of inhibiting crystallization of the dimethyl sulfoxide and inhibiting reduction of cohesion of the adhesive layer, the lower limit of the amount of the medium-chain triglyceride is preferably 0.1% by mass or greater, more preferably 0.5% by mass or greater, yet more preferably 1% by mass or greater, particularly preferably 3% by mass or greater, and the most preferably 5% by mass or greater. The upper limit of the amount of the medium-chain triglyceride is preferably 20% by mass or less, more preferably 15% by mass or less, and yet more preferably 10% by mass or less.

In the present invention, a product that is commercially available for use in pharmaceutical products may be used as the medium-chain triglyceride that is a liquid at room temperature or the medium-chain triglyceride-containing oil or fat that is a liquid at room temperature.

### (f) Filler

The filler may be added for adjusting flexibility of the adhesive layer.

The filler is not particularly limited, and may be appropriately selected according to the intended purpose. Examples of the filler include: silicon compounds, such as silicic anhydride, light anhydrous silicic acid, silicic acid hydrate, and the like; cellulose derivatives, such as ethyl cellulose, methyl cellulose, hydroxypropyl cellulose, hydroxypropyl methyl cellulose, and the like; water-soluble polymers, such as polyvinyl alcohol and the like; aluminum compounds, such as dried aluminum hydroxide gel, hydrous aluminum silicate, and the like; kaolin; titanium oxide; and the like. Among the above-listed examples, light anhydrous silicic acid is preferred in view of improvements of dispersibility and cohesion. The above-listed fillers may be used alone, or in combination.

When the adhesive layer includes the filler, an amount of the filler in the adhesive layer is not particularly limited, and may be appropriately selected according to the intended purpose. The filler may be included in an amount to the extent that high skin permeability, and adequate cohesion and adhesion as a patch can be maintained. Within the above-described amount, the amount of the filler relative to a total amount of adhesive components is preferably 10% by mass or less, more preferably 5% by mass or less, and yet more preferably 2% by mass or less.

### (g1) Tackifier

The “tackifier” is a tackifier that is generally used in a typical field of patches. Examples of the tackifier include rosin-based resins, polyterpene-based resins, coumaron-indene resins, petroleum resins, terpene resins, terpene-phenol resins, saturated alicyclic hydrocarbon resins, and the like. It is important to assure adequate adhesion of the adhesive layer to achieve sufficient efficacy of the drug. If a large amount of the tackifier is added, strong adhesion can be achieved, but an amount of the local anesthetic released from the adhesive layer may be reduced, or skin irritability may be increased. Therefore, the amount of the tackifier in the adhesive layer is preferably 50% by mass or less, more preferably 30% by mass or less, and yet more preferably 10% by mass or less. Particularly preferably, the adhesive layer is free from the tackifier.

### (g2) Polyhydric Alcohol Fatty Acid Monoester

The “polyhydric alcohol fatty acid monoester” is a compound in which one of hydroxyl groups of a polyhydric alcohol, such as ethylene glycol, propylene glycol, glycerin, or the like, forms an ester bond with a fatty acid. The polyhydric alcohol fatty acid monoester contributes to improvement of solubility of the drug without significantly reducing the cohesion of the adhesive base, and has an effect of facilitating absorption.

The polyhydric alcohol constituting the polyhydric alcohol fatty acid monoester is not particularly limited, and may be appropriately selected according to the intended purpose. Examples of the polyhydric alcohol include ethylene glycol, propylene glycol, butylene glycol, glycerin, and the like.

The fatty acid constituting the polyhydric alcohol fatty acid monoester is not particularly limited, and may be appropriately selected according to the intended purpose. The fatty acid is preferably a fatty acid having a carbon number of 8 or greater and 18 or less. Examples of the fatty acid having the carbon number of 8 or greater and 18 or less include capric acid, caprylic acid, myristic acid, palmitic acid, stearic acid, isostearic acid, oleic acid, linoleic acid, and the like.

Specific suitable examples of the polyhydric alcohol fatty acid monoester include propylene glycol monocaprylate, propylene glycol monolaurate, and the like.

When the adhesive layer includes the polyhydric alcohol fatty acid monoester, an amount of the polyhydric alcohol fatty acid monoester in the adhesive layer is not particularly limited, and may be appropriately selected according to the intended purpose. In view of solubility of the local anesthetic and an effect of facilitating absorption, the amount of the polyhydric alcohol fatty acid monoester is preferably 2% by mass or greater, and more preferably 5% by mass or greater.

### (g3) Alcohol-Based Solvent

The “alcohol-based solvent” is not particularly limited, and may be appropriately selected according to the intended purpose. Examples of the alcohol-based solvent include: alcohols having high dissolution capacities, such as benzyl alcohol, diethylene glycol monoethyl ether, and the like; saturated higher aliphatic alcohols that have a carbon number of approximately 12 or greater and approximately 20 or less and are liquids at room temperature, such as lauryl alcohol, isostearyl alcohol, and the like; unsaturated higher aliphatic alcohols that have a carbon number of approximately 12 or greater and approximately 20 or less and are liquids at room temperature, such as oleyl alcohol; and the like.

Among the above-listed examples, benzyl alcohol, diethylene glycol monoethyl ether, lauryl alcohol, or oleyl alcohol is preferred in view of dissolution capacities for the local anesthetic and an effect of facilitating the percutaneous absorption of the local anesthetic.

When the adhesive layer includes the alcohol-based solvent, an amount of the alcohol-based solvent in the adhesive layer is not particularly limited, and may be appropriately selected according to the intended purpose. In view of a dissolution capacity for the local anesthetic and an effect of facilitating absorption, the amount of the alcohol-based solvent is preferably 1% by mass or greater, and more preferably 3% by mass or greater.

### (g4) Ester-Based Solvent

The ester-based solvent is not particularly limited, and may be appropriately selected according to the intended purpose. Examples of the ester-based solvent include diesters between dihydric alcohols and carboxylic acids, esters between polyvalent carboxylic acids and monohydric aliphatic alcohols, carbonate esters, and the like.

Examples of the diesters between the dihydric alcohols and carboxylic acids include diesters between ethylene glycol, propylene glycol, butylene glycol, or the like, and caprylic acid, capric acid, lauric acid, oleic acid, or the like.

Examples of the esters between the polyvalent carboxylic acids and the monohydric aliphatic alcohol include diesters that are liquids at room temperature and are formed between dicarboxylic acid having a carbon number of 2 or greater and 12 or less and a monohydric aliphatic alcohol having a carbon number of 1 or greater and 20 or less, such as diesters of adipic acid that are liquids at room temperature and diesters of sebacic acid that are liquids at room temperature. Examples of the diesters of adipic acid include diethyl adipate, diisopropyl adipate, and the like. Examples of the diester of sebacic acid include diethyl sebacate, diisopropyl sebacate, dioctyldodecyl sebacate, and the like.

Examples of the carbonate esters include cyclic carbonate esters between carbonic acid and diols having a carbon number of 2 or greater and 10 or less, and the like. Examples of the cyclic carbonate esters include ethylene carbonate, propylene carbonate, vinylene carbonate, and the like. Among the above-listed examples, propylene carbonate is preferred.

Among the above-listed ester-based solvents, propylene glycol diesters, adipic acid diesters, sebacic acid diesters, and carbonate esters are preferred, and propylene glycol diesters, diisopropyl adipate, and diethyl sebacate are more preferred.

### (g5) Amide-Based Solvent

The amide-based solvent is not particularly limited, and may be appropriately selected according to the intended purpose. Examples of the amide-based solvent include: pyrrolidones, such as N-methyl-2-pyrrolidone, 2-pyrrolidone, and the like; imidazolinones, such as 1,3-dimethyl-2-imidazolinone and the like; N-substituted toluidine, such as crotamiton and the like; and alkane amides, such as formamide, N-methylformamide, N,N-dimethylformamide, N-methylacetamide, N,N-dimethylacetamide, and N-methylpropanamide, and the like.

Among the above-listed amide-based solvents, N-methyl-2-pyrrolidone, crotamiton, N,N-dimethylformamide, and N,N-dimethylacetamide are preferred, and N-methyl-2-pyrrolidone and crotamiton are more preferred in view of dissolution capacities for the local anesthetic, and improvement of dispersibility and percutaneous absorption of the local anesthetic.

In the present invention, the above-listed alcohol-based solvents, amide-based solvents, and ester-based solvents may be used alone or in combination.

When the adhesive layer includes the alcohol-based solvent, the amide-based solvent, or the ester-based solvent, the lower limit of an amount of the solvent in the adhesive layer is not particularly limited, and may be appropriately selected according to the intended purpose. The lower limit of the amount of the solvent is preferably 0.1% by mass or greater, more preferably 0.5% by mass or greater, and yet more preferably 1% by mass or greater. The upper limit of the amount of the solvent is preferably 30% by mass or less, more preferably 20% by mass or less, and yet more preferably 15% by mass or less.

### (g6) Liquid Organic Acid

The liquid organic acid is not particularly limited, and may be appropriately selected according to the intended purpose. Examples of the liquid organic acid include: aliphatic monocarboxylic acids, such as acetic acid, propionic acid, butyric acid, valeric acid, isovaleric acid, caproic acid, enanthic acid (heptanoic acid), caprylic acid, pelargonic acid (nonanoic acid), and the like; unsaturated aliphatic monocarboxylic acids, such as oleic acid, linoleic acid, arachidonic acid, docosahexaenoic acid, and the like; hydroxycarboxylic acids, such as lactic acid (DL-lactic acid, or a mixture of L-lactic acid and/or D-lactic acid and lactic anhydride) and the like; alkoxy group-substituted liquid carboxylic acids, such as methoxyacetic acid and the like; and sulfonic acid, such as methanesulfonic acid, and the like.

The above-listed liquid organic acids have a function of aiding dissolution of a basic drug so that dispersibility of the basic drug in the adhesive layer can be improved, at the same time as including the basic drug in the adhesive layer at a high concentration. Moreover, the above-listed liquid organic acids have a function of improving percutaneous absorption of the drug. From the above-mentioned standpoint, among the above-listed liquid organic acids, Japanese Pharmacopoeia lactic acid, oleic acid, and isostearic acid are preferred, and Japanese Pharmacopoeia lactic acid is more preferred. The above-listed organic acids may be used alone or in combination.

When the adhesive layer includes the liquid organic acid, the lower limit of an amount of the liquid organic acid in the adhesive layer is not particularly limited, and may be appropriately selected according to the intended purpose. The lower limit of the amount of the liquid organic acid is preferably 0.1% by mass or greater, and more preferably 0.5% by mass or greater. The upper limit of the amount of the liquid organic acid is preferably 20% by mass or less, and more preferably 15% by mass or less.

### (g7) Carboxylic Acid Salt

The carboxylic acid salt is not particularly limited, and may be appropriately selected according to the intended purpose. Examples of the carboxylic acid salt include salts of aliphatic monocarboxylic acids, alicyclic monocarboxylic acids, aliphatic dicarboxylic acids, and the like.

Examples of the aliphatic monocarboxylic acids include: short-chain fatty acids having a carbon number of 2 or greater and 7 or less, such as acetic acid, butyric acid, hexanoic acid, and the like; middle-chain fatty acids having a carbon number of 8 or greater and 11 or less, such as octanoic acid, decanoic acid, and the like; long-chain fatty acids having a carbon number of 12 or greater, such as myristic acid, stearic acid, isostearic acid, oleic acid, and the like; hydroxy monocarboxylic acids, such as glycolic acid, lactic acid, 3-hydroxy butyric acid, mandelic acid, and the like; alkoxy-substituted monocarboxylic acids, such as methoxy acetic acid and the like; and ketomonocarboxylic acids, such as levulinic acid, and the like.

Examples of the alicyclic monocarboxylic acids include alicyclic monocarboxylic acids having a carbon number of 6 or greater and 8 or less, such as cyclohexane carboxylic acid and the like.

Examples of the aliphatic dicarboxylic acids include sebacic acid, adipic acid, malic acid, maleic acid, fumaric acid, and the like.

Examples of the preferred carboxylic acids include long-chain fatty acids having a carbon number of 12 or greater, and hydroxy monocarboxylic acids. Examples thereof include myristic acid, stearic acid, isostearic acid, oleic acid, lactic acid, and the like. Among the above-listed examples, oleic acid or lactic acid is preferred.

Examples of the salts of the carboxylic acid include: alkali metal salts, such as sodium salts, potassium salts, and the like; alkaline earth metal salts, such as calcium salts and the like; and amine salts. In view of easiness in sourcing, stability and an effect of improving percutaneous absorption, sodium salts are preferred.

### (g8) Lactone

The lactone is not particularly limited, and may be appropriately selected according to the intended purpose. Examples of the lactone include five-membered lactones, such as ascorbic acid, isoascorbic acid, and the like.

In the patch of the present invention, the carboxylic acid salt or lactone is preferably sodium oleate, sodium lactate, ascorbic acid, or isoascorbic acid, considering an effect of improving stability of the drug, or an effect of improving percutaneous absorption.

When the adhesive layer includes the carboxylic acid salt or lactone, the lower limit of an amount of the carboxylic acid salt or lactone in the adhesive layer relative to 1 mol of the local anesthetic is not particularly limited, and may be appropriately selected according to the intended purpose. The lower limit of the amount of the carboxylic acid salt or lactone is preferably 0.1 mol or greater. The upper limit of the amount of the carboxylic acid salt or lactone is preferably 5 mol or less, and more preferably 3 mol or less. When the amount of the carboxylic acid salt or lactone is less than 0.1 mol relative to 1 mol of the local anesthetic, a sufficient effect of improving percutaneous absorption may not be obtained. When the amount of the carboxylic acid salt or lactone is greater than 5 mol relative to 1 mol of the local anesthetic, desired physical properties of a preparation, such as adhesion, may not be obtained.

### (g9) Surfactant

The surfactant is not particularly limited, and may be appropriately selected according to the intended purpose. Examples of the surfactant include nonionic surfactants, anionic surfactants, cationic surfactants, and amphoteric surfactants, and the like. Examples of the nonionic surfactant include: polyoxyethylene fatty acid esters, such as polyoxyethylene monolaurate; polyoxyethylene sorbitol fatty acid esters, such as polyoxyethylene sorbitol tetraoleate; polyoxyethylene sorbitan fatty acid esters, such as polyoxyethylene sorbitan monooleate, polyoxyethylene sorbitan monolaurate, and polyoxyethylene sorbitan monopalmitate; sorbitan fatty acid esters, such as sorbitan monolaurate, sorbitan monooleate, sorbitan sesquioleate, and sorbitan trioleate; glycerin fatty acid esters, such as glycerin monooleate, polyoxyethylene castor oil derivatives, and polyoxyethylene hydrogenated castor oil; polyoxyethylene higher aliphatic alcohol ethers, such as polyoxyethylene lauryl ether and polyoxyethylene oleyl ether; polyoxyethylene alkyl phenyl ethers, such as polyoxyethylene nonyl phenyl ether; polyoxyethylene alkyl amino ethers, such as polyoxyethylene lauryl amine and polyoxyethylene oleyl amine; polyoxyethylene-polyoxypropylene copolymers, such as product names of Pluronic (registered trademark) L-31 and Pluronic (registered trademark)L-44; polyglyceryl-based ethers, such as polyglyceryl-3-dioleate; and the like. Examples of the anionic surfactant include sodium alkyl sulfates, such as sodium lauryl sulfate, and the like. Examples of the cationic surfactants include alkyl trimethyl ammonium salts, alkyl dimethyl ammonium salts, and the like. Examples of the amphoteric surfactants include alkyl dimethyl amine oxide, alkyl carboxybetaine, and the like. The above-listed surfactants may be used alone or in combination.

Among the above-listed surfactants, a nonionic surfactant that is a liquid at room temperature is preferred, a sorbitan fatty acid ester that is a liquid at room temperature is more preferred, and sorbitan monolaurate and polyoxyethylene sorbitan monolaurate are particularly preferred for enhancing percutaneous absorption.

When the adhesive layer includes the surfactant, the lower limit of an amount of the surfactant in the adhesive layer is not particularly limited, and may be appropriately selected according to the intended purpose. In view of improvement of skin permeability of the drug, the lower limit of the amount of the surfactant is preferably 0.01% by mass or greater, and more preferably 0.1% by mass or greater. If the amount of the surfactant is large, adhesion may decrease. Therefore, the upper limit of the amount of the surfactant is preferably 10% by mass or less, more preferably 5% by mass or less, yet more preferably 3% by mass or less, particularly preferably 1% by mass or less, and the most preferably 0.5% by mass or less.

(g10) Antioxidant

The antioxidant is not particularly limited, and may be appropriately selected according to the intended purpose. Examples of the antioxidant include dibutylhydroxytoluene, 4-dioxyphenol, tocopherol, tocopherol ester derivatives, EDTA-2Na, rutin, ascorbic acid, N,N-dimethylthiourea, L-cysteine, 1-thioglycerol, 2-mercaptobenzimidazole, and the like. Among the above-listed examples, dibutylhydroxytoluene, tocopherol, or 2-mercaptobenzimidazole is preferred. The above-listed antioxidants may be used alone, or in combination.

When the adhesive layer includes the antioxidant, an amount of the antioxidant in the adhesive layer is not particularly limited, and may be appropriately selected according to the intended purpose. The adhesive layer may include the antioxidant in an amount to the extent that high skin permeability and adequate cohesion and adhesion as a patch can be maintained. Within the above-mentioned amount, the amount of the antioxidant is preferably 10% by mass or less, more preferably 5% by mass or less, and yet more preferably 2% by mass or less.

(g11) Crystallization Inhibitor

The crystallization inhibitor may be added for inhibiting crystallization of the local anesthetic in the adhesive layer.

The crystallization inhibitor is not particularly limited, and may be appropriately selected according to the intended purpose. Examples of the crystallization inhibitor include polyvinyl pyrrolidone, vinyl acetate-vinylpyrrolidone copolymers, polyvinyl caprolactam-polyvinyl acetate-polyethylene glycol graft copolymers, and the like. The above-listed crystallization inhibitors may be used alone, or in combination.

When the adhesive layer includes the crystallization inhibitor, the lower limit of an amount of the crystallization inhibitor in the adhesive layer is not particularly limited, and may be appropriately selected according to the intended purpose. The adhesive layer may include the crystallization inhibitor in an amount to the extent that adhesion as a patch can be maintained. Within the above-described amount, the lower limit of the amount of the crystallization inhibitor is preferably 0.01% by mass or greater, and more preferably 0.1% by mass or greater. The upper limit of the amount of the crystallization inhibitor is preferably 10% by mass or less, and more preferably 5% by mass or less.

(g12) Nonvolatile Hydrocarbon Oil

The nonvolatile hydrocarbon oil is not particularly limited, and may be appropriately selected according to the intended purpose. Examples of the nonvolatile hydrocarbon oil include liquid paraffin, light liquid paraffin, squalene, squalane, and the like. The above-listed examples may be used alone or in combination.

Each of the liquid paraffin and the light liquid paraffin is a mixture of colorless and odorless fluid saturated hydrocarbons. As the liquid paraffin and light liquid paraffin, liquid paraffin and light liquid paraffin each meeting the standard specified by Japanese Pharmacopoeia, US Pharmacopoeia, European Pharmacopoeia, etc. are suitably used.

An amount of the nonvolatile hydrocarbon oil in the adhesive layer is not particularly limited, and may be appropriately selected according to the intended purpose. In view of retention of anesthesia, the amount of the nonvolatile hydrocarbon oil is preferably 50% by mass or less, more preferably 30% by mass or less, yet more preferably 20% by mass or less, yet even more preferably 10% by mass or less, and particularly preferably 5% by mass or less. The most preferably, the adhesive layer is free from the nonvolatile hydrocarbon oil.

### <Other Elements>

The above-mentioned other elements are not particularly limited, and may be appropriately selected according to the intended purpose. Examples of the above-mentioned other elements include a release liner and the like. Specifically, the patch of the present invention may have a structure where the adhesive layer having the above-described configuration is spread over the release liner.

The “release liner” is not particularly limited, and may be appropriately selected according to the intended purpose. As the release liner, release liners generally used for adhesive sheets to be adhered to skin or for transdermal preparations may be used. As the release liner, for example, glassine paper, resin films, aluminum films, polyethylene foam films, polypropylene foam films, or a laminate including two or more of the foregoing may be used. Examples of the resin films include: polyolefins, such as polyethylene, polypropylene, and the like; polyesters, such as polyethylene terephthalate and the like; polystyrene; and the like. Moreover, the release liner subjected to silicone processing, fluororesin processing, embossing, hydrophilic processing, hydrophobic processing, or the like may be used. A thickness of the release liner is typically 10 μm or greater and 200 μm or less, and preferably 15 μm or greater and 150 μm or less.

### (Method for Producing Patch)

A method for producing the patch includes a mixing step of mixing a local anesthetic, a thermoplastic elastomer, a higher fatty acid ester, and dimethyl sulfoxide. The method may further include other steps.

The local anesthetic, thermoplastic elastomer, higher fatty acid ester, and dimethyl sulfoxide are as described above in the description of (Patch).

### <Mixing Step>

The mixing step is not particularly limited, and may be appropriately selected according to the intended purpose. Examples of the mixing step include a method where (a) a local anesthetic serving as a drug, (b) a thermoplastic elastomer, (c) a higher fatty acid ester, and (d) dimethyl sulfoxide are each dissolved or dispersed in a volatile solvent to be mixed. The mixing step can yield a coating liquid for forming an adhesive layer.

The volatile solvent is preferably a volatile solvent that can homogeneously dissolve or disperse the (a) local anesthetic, the (b) thermoplastic elastomer, the (c) higher fatty acid ester, and the (d) dimethyl sulfoxide. Examples of the volatile solvent include: aromatic hydrocarbons, such as toluene and the like; alicyclic hydrocarbons, such as cyclohexane, methyl cyclohexane, and the like; aliphatic hydrocarbons, such as hexane, heptane, and the like; ethers, such as tetrahydrofuran, diethyl ether, t-butyl methyl ether, and the like; ketones, such as acetone, methyl ethyl ketone, methyl isobutyl ketone, and the like; alcohols, such as ethanol, propanol, butanol, and the like; and acetic acid esters, such as ethyl acetate, propyl acetate, isopropyl acetate, butyl acetate, isobutyl acetate, and the like. The above-listed volatile solvents may be used alone, or in combination.

In view of desirable dissolution capacities for each of components constituting the adhesive layer, a single use of or a mixture of two or more selected from the aromatic hydrocarbon, such as toluene and the like, the alicyclic hydrocarbon, such as cyclohexane methyl cyclohexane, and the like, and the aliphatic hydrocarbon, such as hexane, heptane, and the like is more preferred. Alternatively, use of a combination of the aromatic hydrocarbon, such as toluene and the like, the aliphatic hydrocarbon, such as hexane, heptane, and the like, with the acetic acid ester, such as ethyl acetate, propyl acetate, isopropyl acetate, butyl acetate, isobutyl acetate, and the like, is more preferred.

### <Other Steps>

The above-mentioned other steps are not particularly limited, and may be appropriately selected according to the intended purpose. Examples of the above-mentioned other steps include a coating step after the mixing step, a drying step after the coating step, a laminating step for a support and an adhesive layer, a laminating step for a release liner, and the like.

The support, the adhesive layer, and the release liner are as described above.

—Coating Step after the Mixing Step—

Coating of the coating liquid for forming the adhesive layer may be performed, for example, by a commonly used coater, such as a roll coater, a die coater, a gravure roll coater, a reverse roll coater, a kiss roll coater, a dip roll coater, a bar coater, a knife coater, a spray coater, and the like.

—Drying Step after the Coating Step—

Drying of the coating liquid is preferably carried out by heating, for example, at a temperature of approximately 40° C. or higher and approximately 150° C. or lower. A drying temperature, drying duration, and drying method may be adjusted according to a solvent used in the coating liquid, or an amount of the solvent used. A weight of the adhesive layer per unit area after drying may be adjusted according to intended adhesion to skin, and percutaneous absorption performance. As a range where production of the adhesive layer is performed while assuring adhesion to skin, the weight of the adhesive layer per unit area after drying is preferably 10 g/m² or greater and 1,000 g/m² or less, more preferably 20 g/m² or greater and 800 g/m² or less, and yet more preferably 30 g/m² or greater and 600 g/m² or less.

### —Laminating Step for Support and Adhesive Layer—

The laminating step for the support and the adhesive layer is not particularly limited, and may be appropriately selected according to the intended purpose. Examples of the laminating step include a method where the support is pressure-bonded to the adhesive layer to laminate the support and the adhesive layer together.

### —Laminating Step for Release Liner—

The laminating step for the release liner is not particularly limited, and may be appropriately selected according to the intended purpose. Examples of the laminating step include: a step where the coating liquid for forming the adhesive layer is spread over a release liner, a solvent in the coating liquid is dried to laminate an adhesive layer on a surface of the release liner before the laminating step for the support and the adhesive layer (spreading-drying step); a step where the release liner is pressure-bonded to the adhesive layer to laminate the release liner after the laminating step for the support and the adhesive layer; and the like.

### Examples

The present invention will be more concretely described through Examples and Comparative Examples hereinafter, but Examples shall not be construed as to limit the scope of the present invention in any way.

### (Evaluation Method) Skin Permeability Evaluation

Excised abdominal hairless rat skin (Japan SLC, Inc., wistar, male, 5 weeks old) was set in a vertical Franz diffusion cell (product number: TP-8s, produced by Vidrex Company Limited, currently available from PermeGear, Inc.). Each of patches produced in Examples and Comparative Examples was cut out into a circular piece having a diameter of 1.0 cm, and the cut piece was adhered to the rat skin in the Franz diffusion cell. A test was carried out at a temperature of 32′C using a phosphate buffered saline as a receptor fluid. Three hours after the start of the test, part of the receptor fluid was sampled. Cumulative diffusion amounts of the lidocaine and prilocaine that permeated through the rat skin were quantified by HPLC. A patch disclosed in Example 2 of Patent Literature 2 was produced as a control (Comparative Example 1), and the experiments were carried out with reference to the control.

### [Examples 1 to 3 and Comparative Examples 1 to 4] Preparation of Patch

Substances constituting an adhesive layer were weighed and collected according to the formulation presented in Table 1. First, a styrene-isoprene-styrene block copolymer (produced by JSR Corporation) was dissolved in propyl acetate. Then, the substances presented in Table 1 were added, and the resultant mixture was mixed and stirred to prepare a coating liquid for forming an adhesive layer.

Lidocaine was acquired from Nippon Bulk Yakuhin Co., Ltd., prilocaine was acquired from SHIRATORI Pharmaceutical Co., Ltd., and liquid paraffin was acquired from Sonneborn LLC.

The coating liquid was applied onto a polyethylene terephthalate (PET) film (release liner), which had been subjected to silicone processing, in a manner such that a thickness of an adhesive layer after drying was to be approximately 350 μm. After drying the solvent in the applied coating liquid, a PET film (support) was laminated on the surface of the adhesive layer. The resultant laminate was cut into a size of 15 cm×30 cm, thereby obtaining an intended patch.

The skin permeability was evaluated as described above, and a rate of a total amount of the cumulative diffusion amounts of the lidocaine and prilocaine measured three hours after the start of the test relative to a total amount of the cumulative diffusion amounts of the lidocaine and prilocaine in Comparative Example 1 is presented in Table 1.

TABLE 1

| | | | | Comp. | Comp. | Comp. | Comp. |
|---|---|---|---|---|---|---|---|
| | Ex. 1 | Ex. 2 | Ex. 3 | Ex. 1 | Ex. 2 | Ex. 3 | Ex. 4 |
| Lidocaine | 5.0 | 6.0 | 6.5 | 5.0 | 5.0 | 6.0 | 7.5 |
| Prilocaine | 5.0 | 6.0 | 6.5 | 5.0 | 5.0 | 6.0 | 7.5 |
| Styrene-isoprene-styrene | 40.0 | 40.0 | 40.0 | 45.0 | 45.0 | 44.0 | 42.5 |
| copolymer | | | | | | | |
| Octyldodecyl myristate | 48.0 | 46.0 | 45.0 | | | 44.0 | 42.5 |
| Dimethyl sulfoxide | 2.0 | 2.0 | 2.0 | | 2.0 | | |
| Liquid paraffin | | | | 45.0 | 43.0 | | |
| Rate (times) of total | 3.4 | 3.9 | 4.2 | Standard | 3.1 | 1.8 | 2.1 |
| amount of cumulative | | | | | | | |
| diffusion amounts of | | | | | | | |
| lidocaine and prilocaine | | | | | | | |
| 3 hours after start of | | | | | | | |
| test relative to the | | | | | | | |
| total amount of Comp. | | | | | | | |
| Ex. 1 | | | | | | | |

In comparison between Example 1 and Comparative Example 1, Example 1 demonstrated a skin permeability approximately 3.4 times higher than the skin permeability of Comparative Example 1. A skin permeability of EMLA (registered trademark) CREAM was evaluated, and EMLA (registered trademark) CREAM demonstrated a skin permeability approximately 1.2 times higher than the skin permeability of Comparative Example 1. Moreover, Comparative Examples 2 to 4 also demonstrated a skin permeability approximately 2 or more times higher than the skin permeability of Comparative Example 1.

### (Evaluation Method) Evaluation of Anesthesia

The patches were applied to healthy volunteers for 1 hour. Immediately after removing the patch, the application site was pricked with a mandrin to evaluate the degree of the anesthetic potency based on the following criteria. As a control, commercially available EMLA (registered trademark) CREAM was used.

4: 100% of the anesthetic potency (anesthetic potency comparable with the anesthetic potency observed 1 hour after applying EMLA (registered trademark) CREAM) was demonstrated 1 hour after the application.
3: 80% of the anesthetic potency was demonstrated 1 hour after the application.
2: 60% of the anesthetic potency was demonstrated 1 hour after the application.
1: 30% of the anesthetic potency was demonstrated 1 hour after the application.
0: An anesthetic potency was not demonstrated at all.

### [Examples 4 to 6 and Comparative Examples 5 and 6] Preparation of Patch

Substances constituting an adhesive layer were weighed and collected according to the formulation presented in Table 2 to produce each patch in the same manner as in the preparation method of the patch in Examples 1 to 3 and Comparative Examples 1 to 4.

A terpene resin was acquired from ARAKAWA CHEMICAL INDUSTRIES, LTD.

Anesthetic potency of each of the patches of Examples 1 to 6 and Comparative Examples 1 to 6 was evaluated as described above. The anesthetic potency demonstrated one hour after the application is presented in Table 2.

TABLE 2

| | | | | | | | Comp. | Comp. | Comp. | Comp. | Comp. | Comp. |
|---|---|---|---|---|---|---|---|---|---|---|---|---|
| | Ex. 1 | Ex. 2 | Ex. 3 | Ex. 4 | Ex. 5 | Ex. 6 | Ex. 1 | Ex. 2 | Ex. 3 | Ex. 4 | Ex. 5 | Ex. 6 |
| Lidocaine | 5.0 | 6.0 | 6.5 | 5.0 | 6.0 | 6.5 | 5.0 | 5.0 | 6.0 | 7.5 | 5.0 | 5.0 |
| Prilocaine | 5.0 | 6.0 | 6.5 | 5.0 | 6.0 | 6.5 | 5.0 | 5.0 | 6.0 | 7.5 | 5.0 | 5.0 |
| Styrene- | 40.0 | 40.0 | 40.0 | 45.0 | 45.0 | 45.0 | 45.0 | 45.0 | 44.0 | 42.5 | 45.0 | 35.0 |
| isoprene- | | | | | | | | | | | | |
| styrene | | | | | | | | | | | | |
| copolymer | | | | | | | | | | | | |
| Octyldodecyl | 48.0 | 46.0 | 45.0 | 43.0 | 41.0 | 40.0 | | | 44.0 | 42.5 | 45.0 | 30.0 |
| myristate | | | | | | | | | | | | |
| Dimethyl | 2.0 | 2.0 | 2.0 | 2.0 | 2.0 | 2.0 | | 2.0 | | | | |
| sulfoxide | | | | | | | | | | | | |
| Liquid | | | | | | | 45.0 | 43.0 | | | | |
| paraffin | | | | | | | | | | | | |
| Propylene | | | | | | | | | | | | 10.0 |
| glycol | | | | | | | | | | | | |
| monocaprylate | | | | | | | | | | | | |
| Oleyl alcohol | | | | | | | | | | | | 3.0 |
| Diisopropyl | | | | | | | | | | | | 2.0 |
| adipate | | | | | | | | | | | | |
| Terpene resin | | | | | | | | | | | | 10.0 |
| Anesthetic | 2 | 2 | 3 | 2 | 2 | 3 | 0 | 0 | 0 | 0 | 0 | 0 |
| potency | | | | | | | | | | | | |
| demonstrated 1 | | | | | | | | | | | | |
| hour after | | | | | | | | | | | | |
| application | | | | | | | | | | | | |

All of the patches of Examples 1 to 6 demonstrated anesthetic potency one hour after the application. High anesthetic potency was confirmed with the patches of Examples 3 and 6, in which the drug concentration was increased. Conversely, the patches of Comparative Examples 1 to 5 did not demonstrate anesthetic potency at all. Although the patch of Comparative Example 1 had the skin permeability comparable with the EMLA (registered trademark) CREAM, the anesthetic potency was not sufficient enough to acknowledge anesthesia. The patch of Comparative Example 6 was produced with reference to Example 9 of Patent Literature 3. Similar to the patches of other Comparative Examples, anesthetic potency was not demonstrated with the patch of Comparative Example 6.

As presented in Table 1, the skin permeability of Comparative Example 2 is comparable to the skin permeability of Example 1, and Comparative Examples 3 and 4 also achieved the same or better skin permeability compared with the skin permeability of EMLA (registered trademark) CREAM. However, the patches of Comparative Examples 2 to 4 did not demonstrate anesthetic potency. Therefore, it has been assumed that high skin permeability is not sufficient enough to achieve anesthetic potency that ensures anesthesia.

It has been made clear from the results of Examples 1 to 6 and Comparative Examples 3 and 4 that it is important to add a local anesthetic, a thermoplastic elastomer, a higher fatty acid ester, and dimethyl sulfoxide as substances of the patch to achieve anesthetic potency that ensures anesthesia.

### [Examples 7 to 10 and Comparative Examples 7 and 8] Preparation of Patch

Substances constituting an adhesive layer were weighed and collected according to the formulation presented in Table 3 to produce each patch in the same manner as in the preparation method of the patch in Examples 1 to 3 and Comparative Examples 1 to 4.

Sesame oil and light liquid paraffin were acquired from KANEDA Co., Ltd.

Anesthetic potency of each of the patches of Examples 7 to 10 and Comparative Examples 7 to 8 was evaluated as described above. The anesthetic potency demonstrated one hour after the application is presented in Table 3.

TABLE 3

| | | | | | Comp. | Comp. |
|---|---|---|---|---|---|---|
| | Ex. 7 | Ex. 8 | Ex. 9 | Ex. 10 | Ex. 7 | Ex. 8 |
| Lidocaine | 6.5 | 6.5 | 6.5 | 6.5 | 6.5 | 6.5 |
| Prilocaine | 6.5 | 6.5 | 6.5 | 6.5 | 6.5 | 6.5 |
| Styrene-isoprene- | 40.0 | 40.0 | 40.0 | 40.0 | 40.0 | 40.0 |
| styrene copolymer | | | | | | |
| Octyldodecyl myristate | 35.0 | 34.0 | 33.0 | 32.0 | | |
| Dimethyl sulfoxide | 2.0 | 3.0 | 4.0 | 5.0 | 2.0 | 2.0 |
| Sesame oil | 10.0 | 10.0 | 10.0 | 10.0 | 10.0 | 10.0 |
| Liquid paraffin | | | | | 35.0 | |
| Light liquid paraffin | | | | | | 35.0 |
| Anesthetic potency | 3 | 3 | 3 | 3 | 0 | 0 |
| demonstrated | | | | | | |
| 1 hour after | | | | | | |
| application | | | | | | |

Anesthetic potency of the patches of Examples 7 to 10 was evaluated. All of the patches of Examples 7 to 10 demonstrated strong anesthetic potency one hour after the application. Moreover, precipitation of the active ingredient or additives was not confirmed when the patches were stored in a cold place. Conversely, the patches of Comparative Examples 7 and 8, in which the substance of the plasticizer was changed, did not demonstrate anesthetic potency at all.

### [Examples 11 to 16] Preparation of Patch

Substances constituting an adhesive layer were weighed and collected according to the formulation presented in Table 4 to produce each patch in the same manner as in the preparation method of the patch in Examples 1 to 3 and Comparative Examples 1 to 4.

Light anhydrous silicic acid was acquired from NIPPON AEROSIL CO., LTD.

Anesthetic potency of each of the patches of Examples 11 to 16 was evaluated as described above. The anesthetic potency demonstrated one hour after the application is presented in Table 3.

TABLE 4

| | Ex. 11 | Ex. 12 | Ex. 13 | Ex. 14 | Ex. 15 | Ex. 16 |
|---|---|---|---|---|---|---|
| Lidocaine | 7.0 | 7.0 | 7.0 | 3.0 | 3.5 | 8.0 |
| Prilocaine | 7.0 | 7.0 | 7.0 | 3.0 | 3.5 | 8.0 |
| Styrene-isoprene-styrene | 40.0 | 40.0 | 40.0 | 40.0 | 30.0 | 54.0 |
| copolymer | | | | | | |
| Octyldodecyl myristate | 33.0 | 32.9 | 32.4 | 38.0 | 60.0 | 27.0 |
| Dimethyl sulfoxide | 3.0 | 3.0 | 3.0 | 6.0 | 3.0 | 3.0 |
| Sesame oil | 10.0 | 10.0 | 10.0 | 10.0 | | |
| Polyoxyethylene sorbitan | | 0.1 | 0.1 | | | |
| monolaurate | | | | | | |
| Light anhydrous silicic acid | | | 0.5 | | | |
| Anesthetic potency | 3 | 4 | 4 | 2 | 2 | 3 |
| demonstrated 1 hour after | | | | | | |
| application | | | | | | |

All of the patches of Examples 11 to 16 demonstrated the anesthetic potency one hour after the application. Among the patches of Examples 11 to 16, the patches of Examples 12 and 13, in which polyoxyethylene sorbitan monolaurate, or a combination of polyoxyethylene sorbitan monolaurate and light anhydrous silicic acid was added, demonstrated the anesthetic potency comparable to the anesthetic potency of EMLA (registered trademark) CREAM demonstrated one hour after the application.

### [Examples 17 to 22 and Comparative Example 9] Preparation of Patch

Substances constituting an adhesive layer were weighed and collected according to the formulation presented in Table 5 to produce each patch in the same manner as in the preparation method of the patch in Examples 1 to 3 and Comparative Examples 1 to 4.

TABLE 5

| | | | | | | | Comp. |
|---|---|---|---|---|---|---|---|
| | Ex. 17 | Ex. 18 | Ex. 19 | Ex. 20 | Ex. 21 | Ex. 22 | Ex. 9 |
| Lidocaine | 5.0 | 5.0 | 5.0 | 10.0 | 3.0 | 5.0 | 5.0 |
| Prilocaine | 5.0 | 5.0 | 5.0 | 10.0 | 3.0 | | |
| Tetracaine | | | | | | 5.0 | 5.0 |
| Styrene-isoprene-styrene | 40.0 | 40.0 | 40.0 | 40.0 | 40.0 | 40.0 | 40.0 |
| copolymer | | | | | | | |
| Octyldodecyl myristate | | | | 38.0 | 35.0 | 48.0 | |
| Isostearyl isostearate | 48.0 | | | | | | |
| Cetyl 2-ethylhexanoate | | 48.0 | | | | | |
| Hexyldecyl isostearate | | | 48.0 | | | | |
| Liquid paraffin | | | | | | | 48.0 |
| Dimethyl sulfoxide | 2.0 | 2.0 | 2.0 | 1.0 | 9.0 | 2.0 | 2.0 |
| Sesame oil | | | | | 10.0 | | |
| Anesthetic potency | 2 | 2 | 2 | 2 | 2 | 2 | 0 |
| demonstrated 1 hour after | | | | | | | |
| application | | | | | | | |

All of the patches of Examples 17 to 22 demonstrated anesthetic potency one hour after the application. Conversely, the patch of Comparative Example 9, in which the substance of the plasticizer was changed, did not demonstrate anesthetic potency at all.

For example, embodiments of the present invention are as follows.

<1> A patch includes a support and an adhesive layer on the support, where the adhesive layer includes a local anesthetic, a thermoplastic elastomer, a higher fatty acid ester, and dimethyl sulfoxide.

<2> The patch according to <1>, wherein an amount of the higher fatty acid ester is 50 parts by mass or greater and 200 parts by mass or less relative to 100 parts by mass of the thermoplastic elastomer.

<3> The patch according to <1> or <2>, wherein an amount of the higher fatty acid ester in the adhesive layer is 60% by mass or less.

<4> The patch according to any one of <1> to <3>, wherein a carbon number of an ester moiety of the higher fatty acid ester is 12 or greater and 30 or less.

<5> The patch according to any one of <1> to <4>, wherein the thermoplastic elastomer includes a styrene-based block copolymer.

<6> The patch according to <5>, wherein the styrene-based block copolymer is a mixture including a styrene-isoprene-styrene block copolymer and a styrene-isoprene block copolymer.

<7> The patch according to <6>, wherein an amount of the styrene-isoprene block copolymer in the mixture is 50% by mass or greater.

<8> The patch according to <5>, wherein a 25% by mass toluene solution of the styrene-based block copolymer has a viscosity of 800 mPa·s or greater and 1500 mPa·s or less at a solution temperature of 25° C.

<9> The patch according to any one of <1> to <8>, wherein an amount of the dimethyl sulfoxide is 5 parts by mass or greater and 150 parts by mass or less relative to 100 parts by mass of the local anesthetic.

<10> The patch according to any one of <1> to <9>, wherein an amount of the dimethyl sulfoxide in the adhesive layer is 0.5% by mass or greater and 10% by mass or less.

<11> The patch according to any one of <1> to <10>, wherein the adhesive layer includes a medium-chain triglyceride.

<12> The patch according to any one of <1> to <11>, wherein the adhesive layer includes a filler.

<13> The patch according to any one of <1> to <12>, wherein the adhesive layer includes a surfactant.

<14> The patch according to any one of <1> to <13>, wherein the local anesthetic is at least one selected from the group consisting of lidocaine, prilocaine, tetracaine, benzocaine, bupivacaine, mepivacaine, levobupivacaine, and ropivacaine.

<15> The patch according to <14>, wherein the local anesthetic is a mixture including the lidocaine and the prilocaine.

<16> A method for producing the patch according to any one of <1> to <15> includes mixing the local anesthetic, the thermoplastic elastomer, the higher fatty acid ester, and the dimethyl sulfoxide.

This international application claims priority based on Japanese Patent Application No. 2021-196430 filed on Dec. 2, 2021, and Japanese Patent Application No. 2022-179851 filed on Nov. 9, 2022, the entire contents of which are incorporated herein by reference.

1. A patch comprising:
a support; and
an adhesive layer on the support, the adhesive layer including a local anesthetic, a thermoplastic elastomer, a higher fatty acid ester, and dimethyl sulfoxide.

2. The patch according to claim 1,
wherein an amount of the higher fatty acid ester is 50 parts by mass or greater and 200 parts by mass or less relative to 100 parts by mass of the thermoplastic elastomer.

3. The patch according to claim 1,
wherein an amount of the higher fatty acid ester in the adhesive layer is 60% by mass or less.

4. The patch according to claim 1,
wherein a carbon number of an ester moiety of the higher fatty acid ester is 12 or greater and 30 or less.

5. The patch according to claim 1,
wherein the thermoplastic elastomer includes a styrene-based block copolymer.

6. The patch according to claim 5,
wherein the styrene-based block copolymer is a mixture including a styrene-isoprene-styrene block copolymer and a styrene-isoprene block copolymer.

7. The patch according to claim 6,
wherein an amount of the styrene-isoprene block copolymer in the mixture is 50% by mass or greater.

8. The patch according to claim 5,
wherein a 25% by mass toluene solution of the styrene-based block copolymer has a viscosity of 800 mPa-s or greater and 1500 mPa-s or less at a solution temperature of 25° C.

9. The patch according to claim 1,
wherein an amount of the dimethyl sulfoxide is 5 parts by mass or greater and 150 parts by mass or less relative to 100 parts by mass of the local anesthetic.

10. The patch according to claim 1,
wherein an amount of the dimethyl sulfoxide in the adhesive layer is 0.5% by mass or greater and 10% by mass or less.

11. The patch according to claim 1,
wherein the adhesive layer includes a medium-chain triglyceride.

12. The patch according to claim 1,
wherein the adhesive layer includes a filler.

13. The patch according to claim 1,
wherein the adhesive layer includes a surfactant.

14. The patch according to claim 1,
wherein the local anesthetic is at least one selected from the group consisting of lidocaine, prilocaine, tetracaine, benzocaine, bupivacaine, mepivacaine, levobupivacaine, and ropivacaine.

15. The patch according to claim 14,
wherein the local anesthetic is a mixture including the lidocaine and the prilocaine or a mixture of the lidocaine and the tetracaine.

16. A method for producing the patch according to claim 1, the method comprising:
mixing the local anesthetic, the thermoplastic elastomer, the higher fatty acid ester, and the dimethyl sulfoxide.

17. The patch according to claim 1,
wherein the higher fatty acid ester is at least one selected from the group consisting of octyldodecyl myristate, isostearyl isostearate, cetyl 2-ethylhexanoate, and hexyldecyl isostearate.

18. The patch according to claim 1,
wherein the adhesive layer include sesame oil.

19. The patch according to claim 12,
wherein the filler is light anhydrous silicic acid.

20. The patch according to claim 13,
wherein the surfactant is polyoxyethylene sorbitan monolaurate.
